(19) United States
(12) Patent Application Publication
(10) Pub. No.: US 2005/0119616 A1
(43) Pub. Date: Jun. 2, 2005

(54) Catheter bond configuration

(75) Inventors: Richard L. Goodin, Blaine, MN (US); Katherine M. Prindle, Robbinsdale, MN (US)

Correspondence Address: VIDAS, ARRETT & STEINKRAUS, P.A., 6109 BLUE CIRCLE DRIVE, SUITE 2000, MINNETONKA, MN, 55343-9185, US

(73) Assignee: SCIMED LIFE SYSTEMS, INC., Maple Grove, MN (US)

(21) Appl. No.: 11/026,295

(22) Filed: Dec. 30, 2004

Related U.S. Application Data
(62) Division of application No. 10/052,960, filed on Jan. 17, 2002.

(51) Int. Cl. A61M 29/00

(52) U.S. Cl. 604/103

(57) ABSTRACT

A catheter comprises a first component and a second component. The first component has a first outer diameter and the second component has a second outer diameter. At least a portion of the first component being bonded to at least a portion of the second component. When the at least a portion of the first component is bonded to the at least a portion of the second component the first outer diameter is substantially equal to the second outer diameter. A heat shrink material being utilized to bond the at least a portion of the first component to the at least a portion of the second component.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

Medical catheters having a balloon mounted thereon are useful in a variety of medical procedures. Balloon catheters may be used to widen a vessel into which the catheter is inserted by dilating the blocked vessel, such as in an angioplasty procedure. Balloon catheters may also be used to expand and/or seat a medical device such as a stent or graft at a desired position within a body lumen. In all of these applications, fluid under pressure may be supplied to the balloon through an inflation lumen in the catheter, thereby expanding the balloon.

It is essential in the manufacture of balloon catheters to properly seal the balloon to the catheter. The seal must be able to withstand the high pressures to which it is subjected on inflation of the balloon. A poor seal may result in leakage of inflation fluid and inability to achieve the desired pressure or even rapid loss of pressure and deflation of the balloon.

A number of methods for sealing a balloon to a catheter are known in the art. One such method involves the use of a suitable adhesive to bond the balloon to the catheter tube as described, inter alia, in U.S. Pat. No. 4,913,701 to Tower and U.S. Pat. No. 4,943,278 to Euteneuer, et al. The use of adhesives, however, adds to the thickness of the catheter and increase its rigidity at the region of the bonds.

Another such method, where heat fusible materials are employed, involves the application of heat to fuse the balloon to the catheter tube. To that end, resistance heating of copper jaws has been employed to fuse a balloon to a catheter tube. Resistance heating, however, can result in the formation of small, random channels at the balloon-catheter interface, potentially giving rise to undesirable variations in the strength of different bonds. The heat can also cause undesirable crystallization and stiffening of the balloon and catheter material, not only at the bond site, but also in both directions axially of the bond, due to heat conduction through the balloon and the catheter, and heat radiation from the jaws.

A non-contact method for heat sealing a balloon onto a catheter is disclosed in U.S. Pat. No. 4,251,305 to Becker et al. A length of thin tubing is slid over an elongated shaft of the catheter and shrink tubing installed over the thin walled tubing at its ends overlapping the catheter shaft. The shrink tubing is partially shrunk. Lamps emitting energy along the visible and infrared spectra are used to provide radiant energy to form gradually tapering thermoplastic joints that bond the tubing and shaft. This method, nevertheless, suffers from the problem of undesired heat transfer along the catheter and balloon.

Yet another fusion-based method disclosed in U.S. Pat. No. 5,501,759 to Forman involves the use of a beam of laser radiation at a wavelength selected to at least approximately match a wavelength of maximum spectral absorption of the polymeric materials forming the balloon member and body. The polymeric materials are melted by the radiation and then allowed to cool and solidify to form a fusion bond between the catheter tube and the balloon.

Another fusion-based method described in Forman involves the simultaneous use of multiple beams of energy to supply energy at discrete points about the circumference of the balloon and thereby heat the balloon. A single beam is split into multiple beams and the multiple beams directed about the circumference of the balloon via fiber optics.

The various bonding or welding methods for joining a balloon to a catheter as described above or that may be known in the art may be embodied in various configurations. For example, one means of joining components may be accomplished utilizing a lap type engagement. A lap weld or bond involves the overlap of the balloon end over the catheter shaft, or alternatively, the catheter shaft over the end of the balloon. Regardless of the physical orientation of the balloon to the catheter or vice versa the components may be bonded, welded, or other wise engaged together by any of the various methods known or described.

Joining components by lap welding or bonding has the benefit of providing a relatively large engagement surface between components to ensure a secure engagement therebetween. An inherent consequence of lap joining components is an increase in thickness of the catheter at the site of the joining. This increase in thickness may result in a stiffening which may reduce trackability of the catheter device. In addition, the increased thickness adds to the profile of the catheter which may limit the usefulness of the catheter as it may be unable to fit into the narrow confines of certain body vessels.

It would be desirable to provide catheters with a bonding method which provides a secure engagement between components, particularly between a catheter shaft and balloon, wherein the bonding site does not express a thickening nor undesirably increase the profile of the catheter.

All US patents and patent applications and all other publications referenced herein are incorporated herein by reference in their entirety.

### BRIEF SUMMARY OF THE INVENTION

The present invention is directed to several different embodiments. In at least one embodiment the invention may be directed to a unique method and apparatus wherein catheter components may be joined together in a unique manner. Some embodiments of the invention are directed to a method of joining a portion of a catheter shaft to an end of a medical balloon. In such an embodiment the joining method may utilize heat shrinkable tubing to secure the components together without increasing the thickness of the joining area or increasing the profile of the catheter.

Further aspects of the invention will become apparent from the detailed description which follows.

### BRIEF DESCRIPTION OF THE SEVERAL VIEWS OF THE DRAWINGS

A detailed description of the invention is hereafter described with specific reference being made to the drawings in which:

FIG. 1 is a side elevational of a catheter utilizing a PRIOR ART bond configuration between components;

FIG. 2 is a side elevational view of an embodiment of the invention wherein a catheter employs the bond configuration described herein;

FIG. 3 is a depiction of a step in an inventive method for joining catheter components;

FIG. 4 is a depiction of a step in an inventive method for joining catheter components; and

FIG. 5 is a side elevational view of an embodiment of the invention wherein a catheter employs the bond configuration described herein.

### DETAILED DESCRIPTION OF THE INVENTION

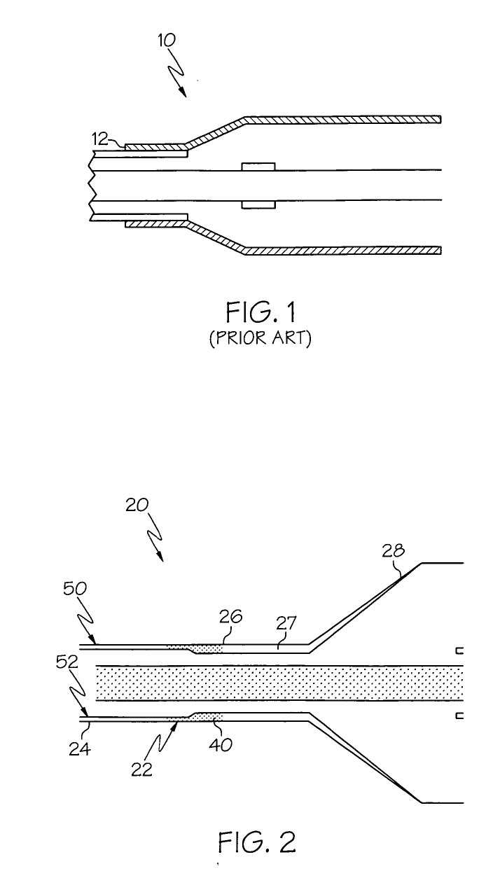

As has been discussed above, the present invention is directed to several different embodiments. At least one embodiment of the invention is directed to a particular type of joining or bond configuration between catheter components, particularly between a balloon end and the catheter shaft. In such an embodiment the bond between components does not appreciably increase the exterior thickness of the catheter shaft or increase the exterior profile of the catheter. In many prior bonding arrangements when components were joined together, such as through the utilization of a lap weld configuration, the overlapping of components resulted in an increase in thickness and profile. An example of a catheter 10 having such a prior lap joining configuration 12 is shown in Prior Art FIG. 1.

While such lap configurations 12 are useful, it is desirous to provide a catheter with a bonding configuration wherein the external diameter of the catheter at the bond is no greater than the diameter of the one or more sections of the catheter immediately adjacent thereto. One example of a catheter 20 employing such an improved bond configuration 22 is shown in FIG. 2.

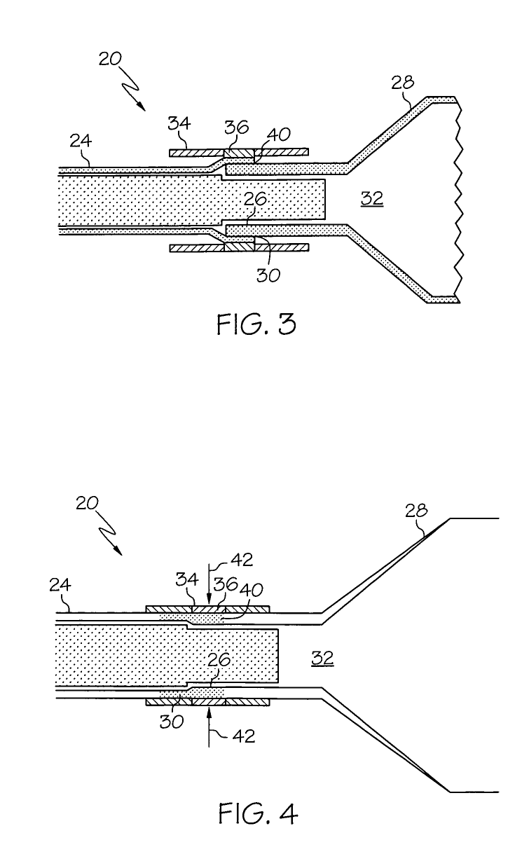

In the embodiment shown in FIG. 2, a catheter 20 includes a catheter shaft 24 which is joined to an end 26 of a waist portion 27 of a medical balloon 28. While the exterior bond configuration 22 between the shaft 24 and end 26 appears to be a continuous extension of tubing, the components have actually been bonded together in the manner depicted in FIGS. 3 and 4.

In FIG. 3, an end 30 of the catheter shaft 24 is disposed about the end 26 of the balloon 28. A tapered or step mandrel 31 may be inserted into the lumen 32 of the catheter 20 to support the shaft 24 and balloon 28 during the joining process. Once the ends 26 and 30 are appropriately positioned, a band 34 of heat shrink material 36 is disposed about the ends 26 and 30.

As is shown in FIG. 3, the mandrel 31 may have a step configuration to allow the overlapped ends 26 and 30 to be pressed together so that the balloon end 26 shares a common outer diameter with the shaft 24 as is shown in FIG. 4.

It should be noted that in the embodiment shown in FIG. 3 the particular arrangement and thicknesses of the ends 26 and 30 are merely exemplary. For example, in the embodiment shown the balloon end 26 has a thickness greater than that of the shaft end 30 prior to bonding. In alternative embodiments of the invention however, the shaft end 30 may be thicker, or the ends 26 and 30 may have equal thicknesses. Furthermore, in alternative embodiments of the invention, the balloon end 26 may be disposed about the shaft end 30 prior to bonding rather than the reverse configuration shown.

The heat shrink material 36 of band 34 may be any heat shrinkable material of suitable characteristics which is configured to press the ends 26 and 30 together when the ends 26 and 30 are subjected to temperatures at or around the melting point(s) of the catheter shaft 24 and/or balloon 28 materials. In at least one embodiment of the invention the band is constructed of a polyethylene material such as polyolefin. An example of a suitable heat shrink material 36 is RNF-100 a heat shrink tubing available from Raychem Corporation. Other examples of heat shrink material include, but are not limited to: Kynar™, nylon, polyvinalchloride, polytetrafluoroethylene, and fluorinated ethylene polymer (FEP).

Once the band 34 is placed over both of the ends 26 and 30, the ends 26 and 30 are heated to their melting points in order to provide the shaft 24 with a smooth bond configuration 22. The temperature to melt the ends 26 and 30 will vary depending on the composition of the catheter shaft 24 and the balloon 28. Typically however, a temperature of about 200 to about 320 degrees Celsius is sufficient to melt any materials which the balloon 28 or catheter shaft 24 may be constructed from.

In the embodiment described above, the bond site 40 may be heated in a variety of different ways. For example, the site 40 may be heated through indirect or direct application of thermal energy, application of laser light of a particular frequency, use of chemical agents to bond and/or heat the ends 26 and 30. In addition, heating and heat shrinking of the bond site 40 may be supplemental to other bonding means such as the aforementioned use of chemical bonding agents, or other joining means as may be know.

While the inventors do want to be limited to a particular theory, it is believed that as the bonding site 40 is heated, the heat shrink band 34 will begin to contract, thereby exerting a constrictive force, indicated by arrows 42, to push the ends 26 and 30 together, such as is shown in FIG. 4. When the temperature is sufficient to melt the material of one or both ends 26 and 30, the ends 26 and 30 will be effectively pushed into one another by the force 42 supplied by band 34. When the band 34 reaches the limit of its shrinkability, or the ends 26 and 30 have been pushed together to a sufficient extent to form the appearance of a single tube of material, such as depicted in FIG. 2, the catheter 20 is allowed to cool and the band 34 may be subsequently removed.

As may be seen in FIG. 2 when the ends 26 and 30 are properly bonded the outer diameter 50 of the catheter 20 is uniform through the shaft 24. While the inner diameter 52 of the catheter 20 does have a step in thickness between shaft 24 and balloon waist 27 this is only a consequence of the greater pre-bonded thickness of the waist 27 relative to the shaft 24.

The method of bonding described above provides for a catheter wherein the thickness of the combined component materials at a bond site will be no greater than the thickest single component material prior to bonding. For example, in the embodiment shown in FIG. 2, the thickness of the combined ends 26 and 30 at the bond site 40 is no greater than the thickness of the end 26.

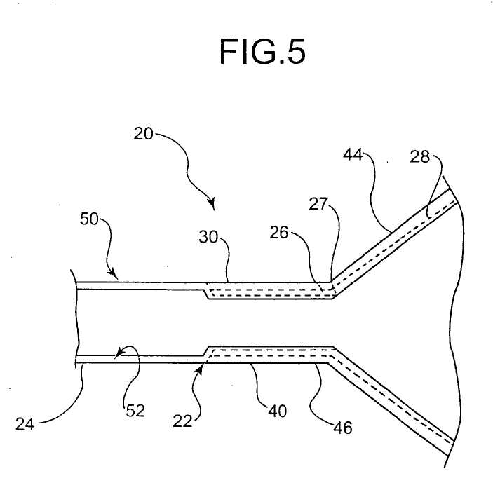

The method show in FIGS. 3 and 4 may also be utilized to join more than two catheter components and may further be utilized to join components other than just the shaft and balloon. For example, in FIG. 5 a catheter 20 is shown wherein the proximal end 26 of a balloon 28 and the proximal end 46 of a sheath, sock or sleeve 44 have been bonded to the distal end of a catheter shaft 24 according to the method described herein. By employing the unique bonding method described herein, the outer diameter 50 of the shaft 24 is uniform, even though two layers of material, in this case from a balloon 28 and sleeve 44, have been bonded to the shaft 24.

Other catheter components, such as for example: sleeves, marker bands, stent retaining hubs, among others, including those previously mentioned above, may all be provided with the unique type of bonding configuration described herein.

In addition to being directed to the specific combinations of features claimed below, the invention is also directed to embodiments having other combinations of the dependent features claimed below and other combinations of the features described above.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

1-17. (canceled)

18. A method of bonding at least two components of a catheter together comprising the following steps:
providing a first catheter component and at least one second catheter component, the first catheter component defining a first layer and the at least one second catheter component defining a second layer, the first layer having a first outside diameter and the second layer having a second outside diameter;
overlapping at least a portion of the first layer and at least a portion of the second layer at least one bonding site;
placing a heat shrinkable band about the at least one bonding site, the heat shrinkable band constructed and arranged to exert a constricting force on the at least a portion of the first layer and the at least a portion of the second layer at the at least one bonding site when the bonding site is heated to a predetermined temperature;
heating the bonding site to cause the heat shrinkable band to force the overlapping at least a portion of the first layer and the at least a portion of the second layer together to form a single layer wherein the first outer diameter is substantially equal to the second outer diameter.

19. The method of claim 18 wherein the first component defines a catheter shaft.

20. The method of claim 18 wherein the at least one second component is selected from at least one member of the group consisting of medical balloons, sleeves, marker bands, stent retaining hubs and any combination thereof.

21. The method of claim 18 wherein the predetermined temperature is the melting point of at least one of the at least a portion of the first component and the at least a portion of the at least one second component.

22. The method of claim 18 wherein the predetermined temperature is about 200 degrees Celsius to about 325 degrees Celsius.

23. The method of claim 18 wherein the heat shrinkable band is constructed from at least one polyethylene based material.

24. The method of claim 18 wherein the heat shrinkable band is constructed from polyolefin.

25. A method of bonding a balloon to a catheter shaft comprising:
providing a catheter shaft having a main shaft portion, a bonding portion distal to the main shaft portion, and a distal end;
providing a balloon, the balloon having a waist portion, the waist portion comprising a proximal end, a bonding portion and a nonbonding portion located distal to the bonding portion;
disposing a portion of the catheter shaft about a portion of the balloon waist portion such that the catheter shaft bonding portion overlaps the balloon bonding portion; and
heating the catheter shaft bonding portion and the balloon waist bonding portion while applying an inward force to the catheter shaft bonding portion, thereby bonding the catheter shaft bonding portion and the balloon bonding portion to form a bonded area, wherein an outer diameter of the bonded area is substantially the same as an outer diameter of the catheter main shaft portion.

26. The method of claim 25, wherein the outer diameter of the bonded area is substantially the same as an outer diameter of the balloon waist nonbonding portion.

27. The method of claim 25, wherein at least a portion of the catheter shaft bonding portion is heated to a temperature ranging from about 200 degrees Celsius to about 325 degrees Celsius.

28. The method of claim 25, wherein at least a portion of the catheter shaft bonding portion is heated to a melting temperature of the catheter shaft bonding portion.

29. The method of claim 25, wherein at least a portion of the balloon waist bonding portion is heated to a melting temperature of the balloon waist bonding portion.

30. The method of claim 25, further comprising:
providing a band of heat-shrinkable material; and
disposing the band of heat-shrinkable material about the catheter shaft bonding portion prior to the heating step.

31. The method of claim 30, wherein the inward force to the catheter shaft bonding portion is applied by the heat-shrinkable material.

32. The method of claim 30, wherein the heat-shrinkable material comprises a polyethylene material.

33. The method of claim 32, wherein the heat-shrinkable material comprises polyolefin.

34. The method of claim 30, wherein the catheter shaft further comprises a catheter lumen extending therethrough, the balloon waist portion further comprises a balloon lumen extending therethrough, and the method further comprises:
providing a mandrel; and
disposing a portion of the mandrel within catheter lumen and the balloon lumen such that the balloon bonding portion is oriented about the portion of the mandrel prior to the heating step.

35. The method of claim 25, wherein the catheter shaft further comprises a catheter lumen extending therethrough, the balloon waist portion further comprises a balloon lumen extending therethrough, and the method further comprises:
providing a mandrel; and
disposing a portion of the mandrel within catheter lumen and the balloon lumen such that the balloon bonding portion is oriented about the portion of the mandrel prior to the heating step.

36. The method of claim 35, wherein the mandrel comprises a first portion and a second portion, the first portion having a first diameter and the second portion having a second diameter.

37. The method of claim 36, wherein the mandrel is disposed such that the first portion is oriented within the catheter main shaft portion and the second portion is oriented within the balloon bonding portion prior to the heating step.
